(19) United States
(12) Patent Application Publication
(10) Pub. No.: US 2005/0161120 A1
(43) Pub. Date: Jul. 28, 2005

(54) Orthopedic and dental endosseous implants and their preparation method

(75) Inventors: Masahiko Inagaki, Aichi (JP); Tetsuya Kameyama, Aichi (JP); Yukiharu Hasegawa, Aichi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Yukiharu Hasegawa, Nagoya-shi (JP); Natl. Inst. of Adv. Indust. Sci. & Tech., Tokyo (JP)

(21) Appl. No.: 10/866,132

(22) Filed: Jun. 14, 2004

(30) Foreign Application Priority Data
Jun. 13, 2003 (JP) 2003-169524

(51) Int. Cl. A61C 8/00; A61F 2/28

(52) U.S. Cl. 148/220; 433/201.1; 623/23.51; 623/23.5; 427/2.27

(57) ABSTRACT

The present invention provides an orthopedic and dental endosseous implant material having a ceramic coating with good adhesion on a metal substrate, and a method for producing this material, and a coating having a gradient composition composed of a ceramic or metal is formed on a ceramic or metal substrate to moderate the residual stress produced by the difference between the coefficients of thermal expansion of the substrate and the coating, thereby increasing the stability of the coating. With this orthopedic and dental endosseous implant material, and the production method thereof, a combination of a metal or ceramic powder having a coefficient of thermal expansion similar to that of the substrate and a metal or ceramic powder having a coefficient of thermal expansion different from that of the substrate is used as the above-mentioned metal or ceramic, a composite composition is formed using this mixture of powders, the metal is nitrided during the formation of a gradient composition, and a nitride layer is formed in the metal in the gradient composition, thereby increasing the stability of the coating.

### BACKGROUND OF THE INVENTION

1. Field of the Invention

The present invention relates to a novel orthopedic and dental endosseous implant material that has excellent initial anchorage to internal bone, and more particularly relates to a novel type of orthopedic and dental endosseous implant material whose biocompatibility has been improved by coating a metal substrate with a calcium phosphate-based ceramic, wherein the metal is nitrided during the formation of a composite composition composed of the metal and the calcium phosphate-based ceramic, which forms a nitride layer in the metal in the composite coating, thereby increasing the strength of the coating.

The present invention is useful in that it provides an orthopedic and dental endosseous implant material which comprises a calcium phosphate-based ceramic firmly bound to a metal substrate, and which has good biocompatibility, and to a method for manufacturing this material, and provides a novel, high-performance orthopedic and dental endosseous implant material, the development of which has been urgently needed in the field of regenerative medicine.

2. Description of the Related Art

Various attempts have been made in the past at imparting bioactivity to a metal substrate by covering it with a calcium phosphate-based ceramic. Ceramics and metals, however, are markedly different in their properties, such as their specific heat, coefficient of thermal expansion, and thermal conductivity, so residual stress occurs at the interface between the ceramic and metal during heating and cooling when the coating is formed, and this results in cracks and other problems in the coating, decreases the adhesion of the coating, and can cause the coating to separate. One possible way to prevent coating separation is to roughen the surface of the material by sandblasting or the like and apply just a thin coating of calcium phosphate-based ceramic, as is seen in orthopedic and dental endosseous implant materials currently used in clinical applications, but this type of method does not offer a fundamental solution to the problem of the residual stress that occurs at the interface between the calcium phosphate-based ceramic and the metal substrate. Also, with a method involving roughening of the material surface by sandblasting or another such treatment, the particles used for the roughening result in contamination, requiring that the material be carefully washed.

Also, as a way to compensate for the drawbacks to composite material having a covering layer whose coefficient of thermal expansion and other such properties differ greatly from those of the substrate, it has been proposed, for example, that the mixing proportions of the metal used for the substrate (or a metal or ceramic having equivalent properties) and of the ceramic that is used in order to provide additional properties that are absent on the surface of the metal substrate be continuously varied so that on the substrate side there is a higher content the metal used for the substrate (or a metal or ceramic having equivalent properties), and on the outer side there is a higher content of the ceramic that is used in order to provide additional properties that are absent on the surface of the metal substrate, thereby producing a gradient functional material. However, it is not good enough merely to compound these materials. In view of this, pre-adding and dispersing other components has been proposed in an effort to impart longer stability and reliability to the ceramic coating of the covering layer (see Japanese Laid-Open Patent Application S62-156938). Nevertheless, adding other components inevitably results in contamination, and the problem with this type of method is that it is not suitable as a way to produce materials for extended use within the living body.

In light of this situation with prior art, the inventors conducted diligent research aimed at completely solving the various problems encountered with prior art and developing an orthopedic and dental endosseous implant material having excellent initial anchorage to internal bone. As a result, they arrived at the present invention upon discovering that the stated object could be achieved by forming a coating having a gradient composition by combining a metal powder with a calcium phosphate-based ceramic powder, and forming a nitride layer in the metal in the gradient composite coating by nitriding the metal during the formation of the gradient composition.

It is an object of the present invention to provide a novel orthopedic and dental endosseous implant material with improved stability and reliability by increasing the adhesion between the substrate and the ceramic coating without adding any components that would result in contamination, and intentionally controlling the bump structure on the surface, and to provide a method for producing this material.

It is a further object of the present invention to provide a method for producing an orthopedic and dental endosseous implant material in which there are no pretreatment steps such as washing, or surface roughening for the purpose of increasing adhesion, as was performed with conventional methods.

It is yet another object of the present invention to provide a method for producing an orthopedic and dental endosseous implant material with which an orthopedic and dental endosseous implant material having good adhesion between the substrate and the coating and having excellent initial anchorage to internal bone can be produced very efficiently in-fewer steps than with a conventional method.

### SUMMARY OF THE INVENTION

To solve the above problems, the present invention is constituted by the following technological means.

(1) An orthopedic and dental endosseous implant material produced by using a metal substrate, a calcium phosphate-based ceramic powder, and a metal powder, and forming on the substrate a coating having a composite composition composed of a calcium phosphate-based ceramic and a metal, wherein:
(a) a coating having a composite composition composed of a metal and a calcium phosphate-based ceramic is formed on the substrate of the orthopedic and dental endosseous implant material to moderate the residual stress produced by the difference between the coefficients of thermal expansion of the substrate and the coating;
(b) the metal in the composite coating has a nitride of that metal formed therein; and
(c) the adhesive strength and stability of the coating are increased by (a) and (b) above, which results in excellent initial anchorage to internal bone.

(2) The orthopedic and dental endosseous implant material according to (1) above, wherein the metal powder has a particle size of anywhere between 10 and 300 μm, and the calcium phosphate-based ceramic powder has a particle size of anywhere between 0.1 and 300 μm.

(3) The orthopedic and dental endosseous implant material according to (1) above, wherein the thickness of the coating is 1 to 1000 μm.

(4) The orthopedic and dental endosseous implant material according to (1) above, wherein a coating is formed having on its surface bumps of intentionally controlled size, depth, shape, layout pattern, and occurrence frequency.

(5) The orthopedic and dental endosseous implant material according to (1) above, wherein a coating having bumps on its surface is formed by depositing a coating on the substrate of the orthopedic and dental endosseous implant material over an area delineated by masking.

(6) The orthopedic and dental endosseous implant material according to (1) above, wherein the minimum width of the indents or protrusions of the irregularities on the coating surface in the horizontal direction with respect to the surface of the implant material is from 10 to 1000 μm.

(7) The orthopedic and dental endosseous implant material according to (1) above, wherein the aspect ratio between the minimum width and maximum width of the indents or protrusions of the irregularities on the coating surface in the horizontal direction with respect to the surface of the implant material is from 1:1 to 1:3000.

(8) The orthopedic and dental endosseous implant material according to (1) above, wherein the height of the irregularities on the coating surface is from 10 to 1000 μm.

(9) The orthopedic and dental endosseous implant material according to (1) above, wherein the occurrence frequency of the irregularities on the coating surface is from 1 to 1000 irregularities per square centimeter.

(10) The orthopedic and dental endosseous implant material according to (1) above, wherein the size, depth, shape, layout pattern, and occurrence frequency of the irregularities on the coating surface are the same everywhere.

(11) The orthopedic and dental endosseous implant material according to (1) above, wherein the size, depth, shape, layout pattern, and occurrence frequency of the irregularities on the coating surface vary with the location.

(12) A method for producing the orthopedic and dental endosseous implant material according to (1) above, comprising:
(a) using a combination of a calcium phosphate-based ceramic powder and a metal powder having a coefficient of thermal expansion similar to that of the substrate;
(b) mixing the calcium phosphate-based ceramic powder and the metal powder having a coefficient of thermal expansion similar to that of the substrate in desired proportions;
(c) varying the mixing proportions so that the proportion of the metal powder having a coefficient of thermal expansion similar to that of the substrate is higher on the substrate side;
(d) forming a coating having a gradient composition by using this powder mixture; and
(e) forming a nitride layer in the metal in the gradient composite coating by nitriding the metal during the formation of the gradient composition.

(13) The method for producing an orthopedic and dental endosseous implant material according to (12) above, wherein a plasma into which nitrogen has been introduced is used to form the nitride layer in the metal in the gradient composite coating.

(14) The method for producing an orthopedic and dental endosseous implant material according to (12) above, wherein the powder is composed of a calcium phosphate-based ceramic and a metal powder having a coefficient of thermal expansion similar to that of the substrate, and the mixing proportions are varied continuously or non-continuously as desired anywhere from 0 to 100% so that the proportion of metal powder with a coefficient of thermal expansion similar to that of the substrate will be higher on the substrate side.

(15The method for producing an orthopedic and dental endosseous implant material according to (12) above, wherein a heat treatment is performed at 200 to 1200° C. after the coating is formed.

(16) The method for producing an orthopedic and dental endosseous implant material according to (12) above, wherein an immersion treatment is performed with a 0 to 300° C. aqueous solution after the coating is formed.

(17) The method for producing an orthopedic and dental endosseous implant material according to (12) above, wherein the organic component of the coating surface is removed with ultraviolet rays, ozone, or a plasma after the coating is formed.

### BRIEF DESCRIPTION OF THE DRAWINGS

FIG. 1 is a conceptual diagram, and is a cross section of the coating having a gradient composition pertaining to Example 1.

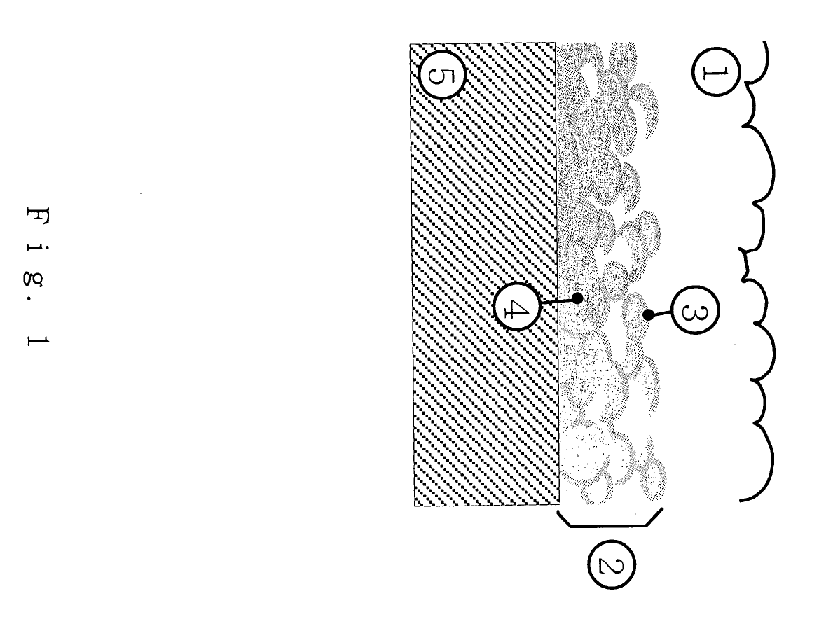

### DESCRIPTION OF THE PREFERRED EMBODIMENTS

The present invention will now be described in further detail.

The present invention relates to a composite material having a gradient composition, and to a method for producing this material, the most salient characteristics of which are that a coating is formed on a substrate made of a metal such as titanium or a titanium alloy by mixing in the desired proportions a metal powder having a coefficient of thermal expansion similar to that of the substrate, with a calcium phosphate-based ceramic powder having a coefficient of thermal expansion different from that of the substrate, varying the mixing proportions thereof continuously or non-continuously so that the proportion of metal powder with a coefficient of thermal expansion similar to that of the substrate will be higher on the substrate side, while melting and depositing this powder mixture by plasma flame spraying or another such method, moderating the difference in the coefficients of thermal expansion between the substrate and the coating, forming a nitride layer in the metal in the composite coating, and markedly increasing the adhesion of the coating. Since no substrate pretreatment, such as roughening the surface of the substrate, is necessary with the present invention, the process of manufacturing a composite material having a gradient composition can be simplified.

With the present invention, titanium, titanium alloys, stainless steel, and so forth can be used favorably as the metal substrate. The powder used to form the coating is a mixture, in any proportions desired, of a powder having a coefficient of thermal expansion similar to that of the substrate and a powder having a coefficient of thermal expansion different from that of the substrate. The mixing proportions are varied continuously or non-continuously. Preferably, with the present invention, the coating is formed by continuously or non-continuously varying the mixing proportions so that the proportion of powder having a coefficient of thermal expansion similar to that of the substrate is higher near the substrate, while the proportion of powder having a coefficient of thermal expansion different from that of the substrate is higher near the coating surface. There are no particular restrictions on the metal powder used as the powder having a coefficient of thermal expansion similar to that of the substrate, but it is preferable to use a powder made of the same material as the substrate.

A favorable example of a powder that can be used as the powder having a coefficient of thermal expansion different from that of the substrate is a powder of a calcium phosphate-based ceramic, but the present invention is not limited to this, and any powder having the same effect can be used. This powder is used in order to impart biocompatibility to the surface of the substrate. As to the method for melting and depositing the mixture of ceramic or metal powders, plasma flame spraying is preferable in that a fairly high temperature is required to melt and deposit the mixture of ceramic or metal powders, form a coating with good adhesion, and nitride the metal powder to form a nitride layer, and in that this method affords greater work efficiency. The plasma flame spraying can involve atmospheric plasma flame spraying, reduced pressure plasma flame spraying, or the like as desired. Types of plasma include high frequency plasma and DC plasma, but a high frequency plasma is preferred because no contamination results from electrode consumption.

The following is a specific example of a favorable method of the present invention. Using titanium powder and hydroxyapatite powder as the above-mentioned powders, for example, these powders are introduced into an apparatus for forming a coating, such as a plasma flame sprayer, with the amounts of the two powders controlled so that the composition of the introduced powders will be 100% titanium and 0% apatite, 70% titanium and 30% apatite, 40% titanium and 60% apatite, and 0% titanium and 100% apatite, and these powders are mixed in the apparatus for forming the coating, or during the course of being introduced into this apparatus, so that a composite coating having a gradient composition is formed, during which time nitrogen gas is introduced into the plasma in order to form a nitride layer in the composite coating. However, the present invention is not limited to these powders or methods, and the types and mix proportions of the above-mentioned powders, the type of the plasma, and the mix proportions of the plasma gas can be appropriately varied according to the targeted product, which should be carried out by a similar method.

With the present invention, forming “a coating having a gradient composition composed of a ceramic or a metal” means coating a substrate while continuously or non-continuously varying the mix proportions of these powders as discussed above, in which case the mix proportions are adjusted so that the proportion of powder having a coefficient of thermal expansion similar to that of the substrate is higher near the substrate, while the proportion of powder having a coefficient of thermal expansion different from that of the substrate is higher near the coating surface, thereby forming a coating of varying composition from the portion in contact with the substrate to the portion at the surface. Forming “a nitride layer in the metal in the gradient composite coating” means forming a nitride layer in this metal, or a diffusion layer in which nitrogen is diffused in the metal, or a layer composed of a mixture of these. The “orthopedic and dental endosseous implant material” referred to in the present invention means a molded article intended for use in the living body. As long as this orthopedic and dental endosseous implant material has the characteristics and safety required for use in the body, there are no particular restrictions on its shape, mode of usage, and so forth.

For example, the shape can be columnar, sheet-form, block-form, wire-form, fibrous, powder-form, or any other shape desired. Favorable examples of the mode of usage include use in products such as artificial hip joint stems, artificial knee joints, artificial spines, artificial vertebrae, bone fillers, bone plates, bone screws, and artificial teeth.

With the above method, a ceramic coating with high reliability and good adhesion to the substrate can be formed without the need for a plurality of steps of substrate surface pretreatment, such as roughening, washing, and drying the surface of the substrate. More specifically, when titanium powder and hydroxyapatite powder are used as the above-mentioned powders, for example, the adhesive strength between the coating and the substrate of the implant material obtained by the above procedure is at least 40 MPa even when the film thickness is 100 μm or greater. The reasons the above-mentioned calcium phosphate coating has such good adhesion to substrates are believed to be that (1) the proportion of the component in the coating having a coefficient of thermal expansion similar to that of the substrate is higher on the substrate side than the proportion of the component having a coefficient of thermal expansion that is different from that of the substrate, (2) the difference in the coefficients of thermal expansion between the substrate and the coating is minimized by varying the composition along a gradient, (3) the composite composition in the coating improves the anchoring effect, and (4) a nitride layer is formed in the metal in the composite composition, which increases the strength of the composite layer, thereby improving adhesion between the coating and its substrate.

### EXAMPLES

The present invention will now be described in specific terms on the basis of examples and comparative examples, but the present invention is not limited to the examples given below.

### Example 1

### Formation of an Apatite/Titanium Composite Coating on a Titanium Substrate, and Formation of a Nitride Layer in the Titanium in the Composite Coating

While varying the mix proportions of titanium powder and hydroxyapatite powder so as to be 100% titanium and 0% apatite, 70% titanium and 30% apatite, 40% titanium and 60% apatite, and 0% titanium and 100% apatite, in that order, these materials were deposited by plasma flame spraying onto a titanium substrate by being introduced into a high frequency plasma of 4 MHz generated at an input of 12 kW, thereby forming a coating of 150 μm. During the formation of this coating, nitrogen was introduced into the plasma to form a nitride layer inside the titanium in the apatite/titanium composite coating. An adhesive strength test was conducted on the obtained product, which revealed the adhesive strength between the substrate and coating to be about 40 MPa.

### Example 2

### Formation of an Apatite/Titanium Composite Coating on a Titanium Alloy Substrate, and Formation of a Nitride Layer in the Titanium in the Composite Coating

While varying the mix proportions of titanium powder and hydroxyapatite powder so as to be 100% titanium and 0% apatite, 70% titanium and 30% apatite, 40% titanium and 60% apatite, and 0% titanium and 100% apatite, in that order, these materials were deposited by plasma flame spraying onto a titanium alloy substrate by being introduced into a high frequency plasma of 4 MHz generated at an input of 17 kW, thereby forming a coating of 150 μm. During the formation of this coating, nitrogen was introduced into the plasma to form a nitride layer inside the titanium in the apatite/titanium composite coating. An adhesive strength test was conducted on the obtained product, which revealed the adhesive strength between the substrate and coating to be about 50 MPa.

### Example 3

### Formation of an Apatite/Titanium Composite Coating on a Titanium Alloy Substrate, and Formation of a Nitride Layer in the Titanium in the Composite Coating

While varying the mix proportions of titanium powder and hydroxyapatite powder so as to be 100% titanium and 0% apatite, 70% titanium and 30% apatite, 40% titanium and 60% apatite, and 0% titanium and 100% apatite, in that order, these materials were deposited by plasma flame spraying onto a titanium alloy substrate by being introduced into a high frequency plasma of 4 MHz generated at an input of 27 kW, thereby forming a coating of 150 μm. During the formation of this coating, nitrogen was introduced into the plasma to form a nitride layer inside the titanium in the apatite/titanium composite coating. An adhesive strength test was conducted on the obtained product, which revealed the adhesive strength between the substrate and coating to be about 65 MPa.

### Comparative Example 1

### Formation of an Apatite Coating on a Relatively Flat Titanium Alloy Substrate

A hydroxyapatite powder was deposited by direct plasma flame spraying onto a titanium substrate by being introduced into a high frequency plasma of 4 MHz generated at an input of 12 kW, and an apatite coating of 100 μm was formed. The material thus obtained was observed to undergo separation of the coating from the substrate after flame spraying, and adequate adhesive strength was not obtained between the substrate and coating.

### Comparative Example 2

### Formation of an Apatite Coating on a Titanium Alloy Substrate on which an Irregular Titanium Coating had been Formed

A titanium powder was deposited by plasma flame spraying onto a titanium substrate by being introduced into a high frequency plasma of 4 MHz generated at an input of 12 kW, and a first cover layer of about 50 μm having irregularities of about 20 μm was formed, after which an apatite coating of 100 μm was flame-sprayed over this first cover layer under the same conditions as in Comparative Example 1. The sample thus obtained was subjected to an adhesive strength test, which revealed the adhesive strength between the substrate and coating to be about 25 MPa.

### Comparative Example 3

### Formation of an Apatite/Titanium Composite Coating on a Titanium Alloy Substrate, with No Formation of a Nitride Layer in the Composite Coating

While varying the mix proportions of titanium powder and hydroxyapatite powder so as to be 100% titanium and 0% apatite, 70% titanium and 30% apatite, 40% titanium and 60% apatite, and 0% titanium and 100% apatite, in that order, these materials were deposited by plasma flame spraying onto a titanium alloy substrate by being introduced into a high frequency plasma of 4 MHz generated at an input of 12 kW, thereby forming a coating of 150 μm. During the formation of this coating, no nitrogen was introduced into the plasma, and no nitride layer was formed inside the titanium in the apatite/titanium composite coating. An adhesive strength test was conducted on the obtained material, which revealed the adhesive strength between the substrate and coating to be about 28 MPa.

### Comparative Example 4

### Formation of an Apatite/Titanium Composite Coating on a Titanium Alloy Substrate, with No Formation of a Nitride Layer in the Composite Coating

While varying the mix proportions of titanium powder and hydroxyapatite powder so as to be 100% titanium and 0% apatite, 70% titanium and 30% apatite, 40% titanium and 60% apatite, and 0% titanium and 100% apatite, in that order, these materials were deposited by plasma flame spraying onto a titanium alloy substrate by being introduced into a high frequency plasma of 4 MHz generated at an input of 17 kW, thereby forming a coating of 150 μm. During the formation of this coating, no nitrogen was introduced into the plasma, and no nitride layer was formed inside the titanium in the apatite/titanium composite coating. An adhesive strength test was conducted on the obtained material, which revealed the adhesive strength between the substrate and coating to be about 18 MPa.

### Example 4

### Cleaning of Coating Surface

A test piece on which an apatite/titanium composite coating had been formed was optically cleaned for 10 minutes using an excimer lamp emitting vacuum ultraviolet light of 172 nm, whereupon the water drop contact angle was about 0°, which represents a marked decrease from the approximate 60° water drop contact angle prior to cleaning. In X-ray photoelectron spectroscopy, the C1s peak produced by contaminating organic components on the surface after optical cleaning was reduced compared to that before cleaning.

### Example 5

### Immersion of Coating in an Aqueous Solution

A test piece on which an apatite/titanium composite coating had been formed was immersed in 20 mM sodium acetate/acetate buffer solution containing 9% table salt at 37° C., and after two weeks of soaking, the elution of calcium ions from the coating had decreased sharply. In the X-ray diffraction pattern of the coating surface after immersion, peaks for calcium oxide, calcium triphosphate, and calcium tetraphosphate (by-products in the coating) disappeared, and the peak for hydroxyapatite increased in size.

### Example 6

### Heat Treatment of Coating

A test piece on which an apatite/titanium composite coating had been formed was heat treated for 1 hour at 600° C., whereupon the crystal phase content of the coating increased from about 50% to about 70%.

### Example 7

### Formation of a Coating Having Surface Irregularities

A coating was formed by performing plasma flame spraying on a titanium alloy substrate using a metal mask in which about 570 circular holes 320 μm in diameter had been made per square centimeter. Protrusions about 250 μm in size were formed on the resulting coating, horizontally with respect to the substrate, with the number of protrusion being about 570 per square centimeter, just as with the mask used. The size of the horizontal irregularities in the horizontal direction with respect to the substrate surface, their shape, and their density could be varied by changing the size, shape, and density of the holes in the mask being used. The depth of the irregularities could be controlled by changing the plasma flame spraying duration.

Reference examples of the present invention will now be described.

### Reference Example 1

An apatite/titanium composite coating having a thickness of 150 μm was formed by plasma flame spraying on a pure titanium rod with a diameter of 2.7 mm to produce a test piece with a diameter of 3 mm and a length of 15 mm. A through-hole with a diameter of 3 mm was made in the main part of the femur of a laboratory animal (dog), and the test piece was inserted. 4 weeks after implantation, the femur in which the test piece had been implanted was excised, and a test was conducted in which the test piece was pulled out of the femur, whereupon the average pull-out strength was about 14.4 MPa.

### Reference Example 2

An apatite/titanium composite coating having a thickness of 150 μm was formed by plasma flame spraying on a pure titanium rod with a diameter of 2.7 mm to produce a test piece with a diameter of 3 mm and a length of 15 mm. This test piece was then heat treated for 1 hour at 600° C. A through-hole with a diameter of 3 mm was made in the main part of the femur of a laboratory animal (dog), and the test piece was inserted. 4 weeks after implantation, the femur in which the test piece had been implanted was excised, and a test was conducted in which the test piece was pulled out of the femur, whereupon the average pull-out strength was about 18.7 MPa.

### Comparative Reference Example 1

A pure titanium rod with a diameter of 3 mm and a length of 15 mm was produced and termed a comparative sample. A through-hole with a diameter of 3 mm was made in the main part of the femur of a laboratory animal (dog), and the test piece was inserted. 4 weeks after implantation, the femur in which the test piece had been implanted was excised, and a test was conducted in which the test piece was pulled out of the femur, whereupon the average pull-out strength was about 1.0 MPa.

As detailed above, the present invention relates to an orthopedic and dental endosseous implant material whose biocompatibility is improved by coating a metal substrate with a calcium phosphate-based ceramic. Effects of the present invention include 1) an orthopedic and dental endosseous implant material with higher coating strength can be produced by nitriding the metal during the formation of a gradient composition composed of a calcium phosphate-based ceramic and a metal, and thereby forming a nitride layer in the metal in the composite coating, 2) regardless of the type and shape of the substrate, a calcium phosphate-based ceramic having a coefficient of thermal expansion that is different from that of the substrate can be formed in a thick coating with good adhesion on an orthopedic and dental endosseous implant substrate, 3) conventional pretreatment steps such as washing or surface roughening for the purpose of increasing the adhesion of the coating can be omitted, which eliminates any contamination that might otherwise be caused by these steps, and 4) the initial anchorage to internal bone can be markedly increased.

1. An orthopedic and dental endosseous implant material produced by using a metal substrate, a calcium phosphate-based ceramic powder, and a metal powder, and forming on the substrate a coating having a composite composition composed of a calcium phosphate-based ceramic and a metal, wherein:
(1) a coating having a composite composition composed of a metal and a calcium phosphate-based ceramic is formed on the substrate of the orthopedic and dental endosseous implant material to moderate the residual stress produced by the difference between the coefficients of thermal expansion of the substrate and the coating;
(2) the metal in the composite coating has a nitride of that metal formed therein; and
(3) the adhesive strength and stability of the coating are increased by (1) and (2) above, which results in excellent initial anchorage to internal bone.

2. The orthopedic and dental endosseous implant material according to claim 1, wherein the metal powder has a particle size of anywhere between 10 and 300 μm, and the calcium phosphate-based ceramic powder has a particle size of anywhere between 0.1 and 300 μm.

3. The orthopedic and dental endosseous implant material according to claim 1, wherein the thickness of the coating is 1 to 1000 μm.

4. The orthopedic and dental endosseous implant material according to claim 1, wherein a coating is formed having on its surface irregularities of intentionally controlled size, depth, shape, layout pattern, and occurrence frequency.

5. The orthopedic and dental endosseous implant material according to claim 1, wherein a coating having irregularities on its surface is formed by depositing a coating on the substrate of the orthopedic and dental endosseous implant material over an area delineated by masking.

6. The orthopedic and dental endosseous implant material according to claim 1, wherein the minimum width of the indents or protrusions of the irregularities on the coating surface in the horizontal direction with respect to the surface of the implant material is from 10 to 1000 μm.

7. The orthopedic and dental endosseous implant material according to claim 1, wherein the aspect ratio between the minimum width and maximum width of the indents or protrusions of the irregularities on the coating surface in the horizontal direction with respect to the surface of the implant material is from 1:1 to 1:3000.

8. The orthopedic and dental endosseous implant material according to claim 1, wherein the height of the irregularities on the coating surface is from 10 to 1000 μm.

9. The orthopedic and dental endosseous implant material according to claim 1, wherein the occurrence frequency of the irregularities on the coating surface is from 1 to 1000 irregularities per square centimeter.

10. The orthopedic and dental endosseous implant material according to claim 1, wherein the size, depth, shape, layout pattern, and occurrence frequency of the irregularities on the coating surface are the same everywhere.

11. The orthopedic and dental endosseous implant material according to claim 1, wherein the size, depth, shape, layout pattern, and occurrence frequency of the irregularities on the coating surface vary with the location.

12. A method for producing the orthopedic and dental endosseous implant material according to claim 1, comprising:
(1) using a combination of a calcium phosphate-based ceramic powder and a metal powder having a coefficient of thermal expansion similar to that of the substrate;
(2) mixing the calcium phosphate-based ceramic powder and the metal powder having a coefficient of thermal expansion similar to that of the substrate in desired proportions;
(3) varying the mixing proportions so that the proportion of the metal powder having a coefficient of thermal expansion similar to that of the substrate is higher on the substrate side;
(4) forming a coating having a gradient composition by using this powder mixture; and
(5) forming a nitride layer in the metal in the gradient composite coating by nitriding the metal during the formation of the gradient composition.

13. The method for producing an orthopedic and dental endosseous implant material according to claim 12, wherein a plasma into which nitrogen has been introduced is used to form the nitride layer in the metal in the gradient composite coating.

14. The method for producing an orthopedic and dental endosseous implant material according to claim 12, wherein the powder is composed of a calcium phosphate-based ceramic and a metal powder having a coefficient of thermal expansion similar to that of the substrate, and the mixing proportions in the powder are varied continuously or non-continuously as desired anywhere from 0 to 100% so that the proportion of the metal powder with a coefficient of thermal expansion similar to that of the substrate is higher on the substrate side.

15. The method for producing an orthopedic and dental endosseous implant material according to claim 12, wherein a heat treatment is performed at 200 to 1200° C. after the coating is formed.

16. The method for producing an orthopedic and dental endosseous implant material according to claim 12, wherein an immersion treatment is performed with a 0 to 300° C. aqueous solution after the coating is formed.

17. The method for producing an orthopedic and dental endosseous implant material according to claim 12, wherein the organic component of the coating surface is removed with ultraviolet rays, ozone, or a plasma after the coating is formed.
